(19) United States
(12) Patent Application Publication
(10) Pub. No.: US 2025/0295836 A1
(43) Pub. Date: Sep. 25, 2025

(54) POLYETHYLENE GLYCOL-FUSION IN NERVE REPAIR

(71) Applicant: BOARD OF REGENTS, THE UNIVERSITY OF TEXAS SYSTEM, Austin, TX (US)

(72) Inventors: George D. BITTNER, Austin, TX (US); Cameron L. GHERGHEREHCHI, Austin, TX (US); Alexa Olivarez, Austin, TX (US); Liwen Zhou, Austin, TX (US)

(73) Assignee: BOARD OF REGENTS, THE UNIVERSITY OF TEXAS SYSTEM, Austin, TX (US)

(21) Appl. No.: 18/860,254

(22) PCT Filed: Apr. 24, 2023
(86) PCT No.: PCT/US2023/066136

§ 371 Date: Oct. 25, 2024

Related U.S. Application Data
(60) Provisional application No. 63/335,430, filed on Apr. 27, 2022.

(51) Int. Cl. A61L 27/36

(52) U.S. Cl. CPC A61L 27/3687; A61L 27/3604; A61L 2430/32

(57) ABSTRACT

The disclosure relates to methods of nerve repair utilizing polyethylene glycol (PEG) to fuse severed axons after injury. This approach does not depend upon the natural regeneration of injured nerves and thus can be applied to spinal cord injuries. These methods can re-establish action potential conduction in damaged spinal cords in vivo.

### PRIORITY CLAIM

This application claims benefit of priority to U.S. Provisional Application Ser. No. 63/335,430, filed Apr. 27, 2022, the entire contents of which are hereby incorporated by reference.

### BACKGROUND

### I. Field

The present disclosure relates to the fields of medicine and neurobiology. More particularly, it addresses the need for improved methods of repairing spinal cord injuries by providing PEG-fused peripheral nerves as graft material and in spinal cord nerve regeneration.

### II. Related Art

The spinal cord transmits sensory reception from the peripheral nervous system. It also conducts motor information to the body's skeletal muscles, cardiac muscles, smooth muscles, and glands. There are 31 pairs of spinal nerves along the spinal cord, all of which consist of both sensory and motor neurons. The spinal cord is protected by vertebrae and connects the peripheral nervous system to the brain, and it acts as a “minor” coordinating center. Unlike in the peripheral nervous system, neuroregeneration in the central nervous system is generally considered not possible.

Any type of traumatic injury to the spinal cord can result in a wide spectrum of disabilities in a person. Depending on the section of the spinal cord that suffers the trauma, the outcome may be anticipated. Infectious diseases may also affect the spinal cord directly. Generally, an infection is a disease that is caused by the invasion of a microorganism or virus. Degenerative spinal disorders involve a loss of function in the spine. Pressure on the spinal cord and nerves may be associated with herniation or disc displacement.

In the United States, about 12,000 people a year survive a spinal cord injury (SCI). The most commonly affected group are young adult males. SCI has seen great improvements in its care since the middle of the 20th century. Research into potential treatments includes stem cell implantation, hypothermia, engineered materials for tissue support, epidural spinal stimulation, and wearable robotic exoskeletons. However, SCI remains a devastating and difficult to treat condition, especially given the inability of central nervous system tissues to naturally regenerate. The expense associated with the care of permanently disabled patients is quite high. As such, improved methods of reestablishing nerve signal transmission following SCI would have a large impact in the treatment of patients with such injuries.

### SUMMARY

Thus, in accordance with the present disclosure, there is provided a method of repairing an injury to a spinal cord nerve in a subject comprising (a) providing a peripheral nerve graft or a spinal tract nerve graft; (b) treating said nerve graft with a membrane fusogen, such as polyethylene glycol (PEG); and (c) suturing the fusogen-treated nerve graft to the damaged spinal cord such that the fusogen-treated nerve graft bridges or span a region of spinal cord damage or a gap in said spinal cord. The fusogen-treated nerve graft may bridge around said region of damaged spinal cord or may span a gap in said spinal cord, optionally wherein said method further comprises remove damaged spinal cord tissue to create said gap. The nerve graft may be an allograft, an autograft or a xenograft. The peripheral nerve graft may derived from a sensory nerve, motor nerve, or a mixed nerve. The fusogen may be a PEG solution.

The subject may be a human subject or a non-human mammalian subject. The spinal cord injury may be an acute injury, a chronic injury, an injury to the cervical spinal cord, an injury to the thoracic spinal cord, an injury to the lumbar spinal cord, an injury to the sacral spinal cord, an injury to the cervical and thoracic spinal cord, an injury to the thoracic and lumbar spinal cord, or an injury to the lumbar and sacral spinal cord. The spinal cord injury may be a contusion spinal cord injury, a transecting spinal cord injury, such as a partial transecting spinal cord injury or a complete transecting spinal cord injury.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Exposure of the spinal cord through a dorsal laminectomy. Prior to injury (left) the spinal cord can be clearly seen with intact vasculature. After a contusion injury, the spinal cord immediately swells with blood and obscures clear visualization. This immediate hemorrhaging most likely interferes with the ability of PEG-fusion to reconnect severed axons after severe contusion-type injuries. The use of peripheral nerve grafts and sharp transection injuries will allow us to bypass this limitation.

FIG. 2. Schematic of the “Bridging” and “Spanning” method of peripheral nerve graft insertion. In the “bridging” approach, nerve grafts are sutured dorsal to the spinal cord, and rostral and caudal to the injury. In the “spanning” approach, the damaged tissue is removed from the spinal cord to create a gap, and nerve grafts are inserted directly into the spinal column and sutured the spinal cord.

FIG. 3. Intraoperative images of the bridging repair. The nerve graft is inserted into the spinal cord through a small incision in the dura and secured with sutures (left). Black dotted lines outline the peripheral nerve graft, white dotted lines outline the spinal cord, and the black arrows point to sutured (right).

FIG. 4. Intraoperative images of the spanning repair. Damaged spinal tissue is removed to create a visible gap (left). Peripheral nerve grafts are inserted directly into the gap and sutured into place (right).

FIG. 5. Preliminary electrophysiological results. The inventors have shown that stimulation of the peripheral nerve graft (left) elicits an action potential recording in the spinal cord, indicating that the nerve graft is functionally connected to axons in the spinal cord. They have also shown that stimulation of the nerve graft elicits twitches and muscle action potential recording in surrounding musculature (right). To date, they have successfully measured re-establishment of action potential conduction in vivo in 4/5 animals using the “bridging” repair, and 3/4 in the “spanning repair. In addition to the in vivo methods, the inventors are currently working to develop an ex vivo testing procedure using a double sucrose gap chamber to get more detailed electrophysiological measures.

FIG. 6. Tests used to determine recovery of spinal cord function. The inventors will use the BBB locomotor test (left) to determine motor function, and the Von Frey hair test (right) to determine sensory function. The BBB test is an open-field test that looks at many aspects of locomotor function: stepping with or without weight support, trunk stability, paw rotation, tail position, toe clearance, plantar stepping, etc. The Von Frey test measures sensory function by applying filaments of varying force to elicit a paw withdrawal response indicating sensory function.

FIGS. 7A-B. (FIG. 7A) Line graph of BBB locomotor scores from 1-8 weeks post-operatively. (FIG. 7B) Bar graph of BBB scores at 8 weeks post-operatively. A score of 21 indicates completely normal function, while a score of 0 indicates complete hindlimb paralysis. Sham and Unoperated animals typically had scores of 21 throughout the study with no significant impairments observed at any time. Complete transection without graft or PEG-fusion repair resulted in an average score of 3±0.8 at 8 weeks PO, indicating extensive movement of 2/3 joints (hip, knee, ankle). Transection with bridge repair and no PEG had similar results (3.3±0.5). Transection with PEG-fused bridge repair resulted in an average score of 5.3±1.0.5, indicating some movement of all three joints, with one animal showing extensive movement of all 3 joints (BBB score of 7). Transection with spanning graft repair (with and without PEG) showed BBB scores ˜1, indicating slight movement of 1 or 2 joints.

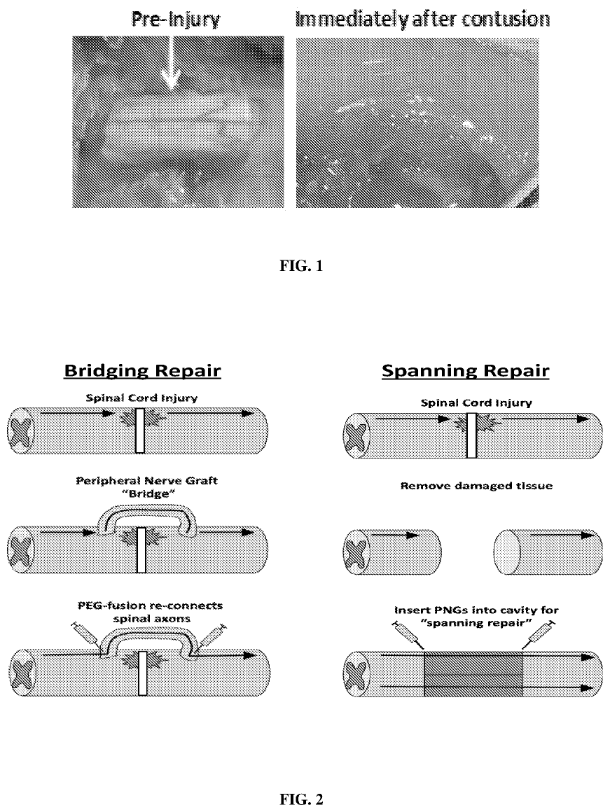

### DETAILED DESCRIPTION

The inventors developed a method of spinal nerve repair utilizing polyethylene glycol (PEG) to fuse severed or damaged axons after injury. This technique does not depend upon the natural regeneration of injured nerves and thus has great potential in spinal cord injuries where there is no regeneration to facilitate natural recovery. The experiments reported herein show that PEG-fusion of peripheral nerve grafts can re-establish conductivity in injured spinal cord nerves. These and other aspects of the disclosure are set out in detail below.

### I. Spinal Cord Nerve Injuries

Nerve injury is any injury to nervous tissue. There is no single classification system that can describe all the many variations of nerve injuries. Usually, peripheral nerve injuries are classified in five stages, based on the extent of damage to both the nerve and the surrounding connective tissue, since supporting glial cells may be involved. Central nervous system disorders include a variety of neurological disorders that affect the structure or function of the brain or spinal cord, which collectively form the central nervous system (CNS).

The spinal cord transmits sensory reception from the peripheral nervous system. It also conducts motor information to the body's skeletal muscles, cardiac muscles, smooth muscles, and glands. There are 31 pairs of spinal nerves along the spinal cord, all of which consist of both sensory and motor neurons. The spinal cord is protected by vertebrae and connects the peripheral nervous system to the brain, and it acts as a “minor” coordinating center. Unlike in the peripheral nervous system, neuroregeneration in the central nervous system is generally considered not possible.

A spinal cord injury (SCI) is damage to the spinal cord that causes temporary or permanent changes in its function. Symptoms may include loss of muscle function, sensation, or autonomic function in the parts of the body served by the spinal cord below the level of the injury. Injury can occur at any level of the spinal cord and can be complete, with a total loss of sensation and muscle function at lower sacral segments, or incomplete, meaning some nervous signals are able to travel past the injured area of the cord up to the S4-5 spinal cord segments. Depending on the location and severity of damage, the symptoms vary, from numbness to paralysis, including bowel or bladder incontinence. Long term outcomes also range widely, from full recovery to permanent tetraplegia (also called quadriplegia) or paraplegia. Complications can include muscle atrophy, loss of voluntary motor control, spasticity, pressure sores, infections, and breathing problems.

In most cases, the damage results from physical trauma such as car accidents, gunshot wounds, falls, or sports injuries, but it can also result from nontraumatic causes such as infection, insufficient blood flow, and tumors. Just over half of injuries affect the cervical spine, while 15% occur in each of the thoracic spine, border between the thoracic and lumbar spine, and lumbar spine alone. Diagnosis is typically based on symptoms and medical imaging.

Efforts to prevent SCI include individual measures such as using safety equipment, societal measures such as safety regulations in sports and traffic, and improvements to equipment. Treatment starts with restricting further motion of the spine and maintaining adequate blood pressure. Corticosteroids have not been found to be useful. Other interventions vary depending on the location and extent of the injury, from bed rest to surgery. In many cases, spinal cord injuries require long-term physical and occupational therapy, especially if it interferes with activities of daily living.

Spinal cord injury can be traumatic or nontraumatic and can be classified into three types based on cause: mechanical forces, toxic, and ischemic (from lack of blood flow). The damage can also be divided into primary and secondary injury: the cell death that occurs immediately in the original injury, and biochemical cascades that are initiated by the original insult and cause further tissue damage. These secondary injury pathways include the ischemic cascade, inflammation, swelling, cell suicide, and neurotransmitter imbalances. They can take place for minutes or weeks following the injury.

At each level of the spinal column, spinal nerves branch off from either side of the spinal cord and exit between a pair of vertebrae, to innervate a specific part of the body. The area of skin innervated by a specific spinal nerve is called a dermatome, and the group of muscles innervated by a single spinal nerve is called a myotome. The part of the spinal cord that was damaged corresponds to the spinal nerves at that level and below. Injuries can be cervical 1-8 (C1-C8), thoracic 1-12 (T1-T12), lumbar 1-5 (L1-L5), or sacral (S1-S5). A person's level of injury is defined as the lowest level of full sensation and function. Paraplegia occurs when the legs are affected by the spinal cord damage (in thoracic, lumbar, or sacral injuries), and tetraplegia occurs when all four limbs are affected (cervical damage).

SCI is also classified by the degree of impairment. The International Standards for Neurological Classification of Spinal Cord Injury (ISNCSCI), published by the American Spinal Injury Association (ASIA), is widely used to document sensory and motor impairments following SCI. It is based on neurological responses, touch and pinprick sensations tested in each dermatome, and strength of the muscles that control key motions on both sides of the body. Muscle strength is scored on a scale of 0-5 according to the table on the right, and sensation is graded on a scale of 0-2:0 is no sensation, 1 is altered or decreased sensation, and 2 is full sensation. Each side of the body is graded independently.

In a “complete” spinal injury, all functions below the injured area are lost, whether or not the spinal cord is severed. An “incomplete” spinal cord injury involves preservation of motor or sensory function below the level of injury in the spinal cord. To be classed as incomplete, there must be some preservation of sensation or motion in the areas innervated by S4 to S5, e.g., voluntary external anal sphincter contraction. The nerves in this area are connected to the very lowest region of the spinal cord and retaining sensation and function in these parts of the body indicates that the spinal cord is only partially damaged. Incomplete injury by definition includes a phenomenon known as sacral sparing: some degree of sensation is preserved in the sacral dermatomes, even though sensation may be more impaired in other, higher dermatomes below the level of the lesion. Sacral sparing has been attributed to the fact that the sacral spinal pathways are not as likely as the other spinal pathways to become compressed after injury due to the lamination of fibers within the spinal cord.

Spinal cord injury without radiographic abnormality exists when SCI is present but there is no evidence of spinal column injury on radiographs. Spinal column injury is trauma that causes fracture of the bone or instability of the ligaments in the spine; this can coexist with or cause injury to the spinal cord, but each injury can occur without the other. Abnormalities might show up on magnetic resonance imaging (MRI), but the term was coined before MRI was in common use.

Central cord syndrome, almost always resulting from damage to the cervical spinal cord, is characterized by weakness in the arms with relative sparing of the legs, and spared sensation in regions served by the sacral segments. There is loss of sensation of pain, temperature, light touch, and pressure below the level of injury. The spinal tracts that serve the arms are more affected due to their central location in the spinal cord, while the corticospinal fibers destined for the legs are spared due to their more external location. The most common of the incomplete SCI syndromes, central cord syndrome usually results from neck hyperextension in older people with spinal stenosis. In younger people, it most commonly results from neck flexion. The most common causes are falls and vehicle accidents; however other possible causes include spinal stenosis and impingement on the spinal cord by a tumor or vertebral disk.

Anterior cord syndrome, due to damage to the front portion of the spinal cord or reduction in the blood supply from the anterior spinal artery, can be caused by fractures or dislocations of vertebrae or herniated disks. Below the level of injury, motor function, pain sensation, and temperature sensation are lost, while sense of touch and proprioception (sense of position in space) remain intact. These differences are due to the relative locations of the spinal tracts responsible for each type of function.

Brown-Séquard syndrome occurs when the spinal cord is injured on one side much more than the other. It is rare for the spinal cord to be truly hemisected (severed on one side), but partial lesions due to penetrating wounds (such as gunshot or knife wounds) or fractured vertebrae or tumors are common. On the ipsilateral side of the injury (same side), the body loses motor function, proprioception, and senses of vibration and touch. On the contralateral (opposite side) of the injury, there is a loss of pain and temperature sensations.

Posterior cord syndrome, in which just the dorsal columns of the spinal cord are affected, is usually seen in cases of chronic myelopathy but can also occur with infarction of the posterior spinal artery. This rare syndrome causes the loss of proprioception and sense of vibration below the level of injury while motor function and sensation of pain, temperature, and touch remain intact. Usually posterior cord injuries result from insults like disease or vitamin deficiency rather than trauma. Tabes dorsalis, due to injury to the posterior part of the spinal cord caused by syphilis, results in loss of touch and proprioceptive sensation.

Conus medullaris syndrome is an injury to the end of the spinal cord, located at about the T12-L2 vertebrae in adults. This region contains the S4-S5 spinal segments, responsible for bowel, bladder, and some sexual functions, so these can be disrupted in this type of injury. In addition, sensation and the Achilles reflex can be disrupted. Causes include tumors, physical trauma, and ischemia.

Cauda equina syndrome (CES) results from a lesion below the level at which the spinal cord splits into the cauda equina, at levels L2-S5 below the conus medullaris. Thus, it is not a true spinal cord syndrome since it is nerve roots that are damaged and not the cord itself; however, it is common for several of these nerves to be damaged at the same time due to their proximity. CES can occur by itself or alongside conus medullaris syndrome. It can cause low back pain, weakness or paralysis in the lower limbs, loss of sensation, bowel and bladder dysfunction, and loss of reflexes. Unlike in conus medullaris syndrome, symptoms often occur on only one side of the body. The cause is often compression, e.g., by a ruptured intervertebral disk or tumor. Since the nerves damaged in CES are actually peripheral nerves because they have already branched off from the spinal cord, the injury has better prognosis for recovery of function: the peripheral nervous system has a greater capacity for healing than the central nervous system.

Complications of spinal cord injuries include pulmonary edema, respiratory failure, neurogenic shock, and paralysis below the injury site. In the long term, the loss of muscle function can have additional effects from disuse, including atrophy of the muscle. Immobility can lead to pressure sores, particularly in bony areas, requiring precautions such as extra cushioning and turning in bed every two hours (in the acute setting) to relieve pressure. In the long term, people in wheelchairs must shift periodically to relieve pressure. Another complication is pain, including nociceptive pain (indication of potential or actual tissue damage) and neuropathic pain, when nerves affected by damage convey erroneous pain signals in the absence of noxious stimuli. Spasticity, the uncontrollable tensing of muscles below the level of injury, occurs in 65-78% of chronic SCI. It results from lack of input from the brain that quells muscle responses to stretch reflexes. It can be treated with drugs and physical therapy. Spasticity increases the risk of contractures (shortening of muscles, tendons, or ligaments that result from lack of use of a limb); this problem can be prevented by moving the limb through its full range of motion multiple times a day. Another problem lack of mobility can cause is loss of bone density and changes in bone structure. Loss of bone density (bone demineralization), thought to be due to lack of input from weakened or paralyzed muscles, can increase the risk of fractures. Conversely, a poorly understood phenomenon is the overgrowth of bone tissue in soft tissue areas, called heterotopic ossification. It occurs below the level of injury, possibly as a result of inflammation, and happens to a clinically significant extent in 27% of people.

People with SCI are at especially high risk for respiratory and cardiovascular problems, so hospital staff must be watchful to avoid them. Respiratory problems (especially pneumonia) are the leading cause of death in people with SCI, followed by infections, usually of pressure sores, urinary tract infections and respiratory infections. Pneumonia can be accompanied by shortness of breath, fever, and anxiety.

Another potentially deadly threat to respiration is deep venous thrombosis (DVT), in which blood forms a clot in immobile limbs; the clot can break off and form a pulmonary embolism, lodging in the lung and cutting off blood supply to it. DVT is an especially high risk in SCI, particularly within 10 days of injury, occurring in over 13% in the acute care setting. Preventative measures include anticoagulants, pressure hose, and moving the patient's limbs. The usual signs and symptoms of DVT and pulmonary embolism may be masked in SCI cases due to effects such as alterations in pain perception and nervous system functioning.

Urinary tract infection (UTI) is another risk that may not display the usual symptoms (pain, urgency, and frequency); it may instead be associated with worsened spasticity. The risk of UTI, likely the most common complication in the long term, is heightened by use of indwelling urinary catheters. Catheterization may be necessary because SCI interferes with the bladder's ability to empty when it gets too full, which could trigger autonomic dysreflexia or damage the bladder permanently. The use of intermittent catheterization to empty the bladder at regular intervals throughout the day has decreased the mortality due to kidney failure from UTI in the first world, but it is still a serious problem in developing countries.

An estimated 24-45% of people with SCI suffer from depression, and the suicide rate is as much as six times that of the rest of the population. The risk of suicide is worst in the first five years after injury. In young people with SCI, suicide is the leading cause of death. Depression is associated with an increased risk of other complications such as UTI and pressure ulcers that occur more when self-care is neglected.

Spinal cord injuries are most often caused by physical trauma. Forces involved can be hyperflexion (forward movement of the head); hyperextension (backward movement); lateral stress (sideways movement); rotation (twisting of the head); compression (force along the axis of the spine downward from the head or upward from the pelvis); or distraction (pulling apart of the vertebrae). Traumatic SCI can result in contusion, compression, or stretch injury. It is a major risk of many types of vertebral fracture. Pre-existing asymptomatic congenital anomalies can cause major neurological deficits, such as hemiparesis, to result from otherwise minor trauma.

In the U.S., Motor vehicle accidents are the most common cause of SCIs; second are falls, then violence such as gunshot wounds, then sports injuries. In some areas, falls are more common, even surpassing vehicle crashes as the leading cause of SCI. The rates of violence-related SCI depend heavily on place and time. Of all sports-related SCIs, shallow water dives are the most common cause; winter sports and water sports have been increasing as causes while association football and trampoline injuries have been declining. Hanging can cause injury to the cervical spine, as may occur in attempted suicide. Military conflicts are another cause and, when they occur, they are associated with increased rates of SCI. Another potential cause of SCI is iatrogenic injury, caused by an improperly done medical procedure such as an injection into the spinal column.

SCI can also be of a nontraumatic origin. Non-traumatic lesions cause anywhere from 30 to 80% of all SCI; the percentage varies by locale, influenced by efforts to prevent trauma. Developed countries have higher percentages of SCI due to degenerative conditions and tumors than developing countries. In developed countries, the most common cause of non-traumatic SCI is degenerative diseases, followed by tumors; in many developing countries the leading cause is infection such as HIV and tuberculosis. SCI may occur in intervertebral disc disease, and spinal cord vascular disease. Spontaneous bleeding can occur within or outside of the protective membranes that line the cord, and intervertebral disks can herniate. Damage can result from dysfunction of the blood vessels, as in arteriovenous malformation, or when a blood clot becomes lodged in a blood vessel and cuts off blood supply to the cord. When systemic blood pressure drops, blood flow to the spinal cord may be reduced, potentially causing a loss of sensation and voluntary movement in the areas supplied by the affected level of the spinal cord. Congenital conditions and tumors that compress the cord can also cause SCI, as can vertebral spondylosis and ischemia. Multiple sclerosis is a disease that can damage the spinal cord, as can infectious or inflammatory conditions such as tuberculosis, herpes zoster or herpes simplex, meningitis, myelitis, and syphilis.

The first stage in the management of a suspected spinal cord injury is geared toward basic life support and preventing further injury: maintaining airway, breathing, and circulation and restricting further motion of the spine. In the emergency setting, most people who has been subjected to forces strong enough to cause SCI are treated as though they have instability in the spinal column and have spinal motion restricted to prevent damage to the spinal cord. Injuries or fractures in the head, neck, or pelvis as well as penetrating trauma near the spine and falls from heights are assumed to be associated with an unstable spinal column until it is ruled out in the hospital. High-speed vehicle crashes, sports injuries involving the head or neck, and diving injuries are other mechanisms that indicate a high SCI risk. Since head and spinal trauma frequently coexist, anyone who is unconscious or has a lowered level of consciousness as a result of a head injury is spinal motion restricted.

A rigid cervical collar is applied to the neck, and the head is held with blocks on either side and the person is strapped to a backboard. Extrication devices are used to move people without excessively moving the spine if they are still inside a vehicle or other confined space. The use of a cervical collar has been shown to increase mortality in people with penetrating trauma and is thus not routinely recommended in this group.

Modern trauma care includes a step called clearing the cervical spine, ruling out spinal cord injury if the patient is fully conscious and not under the influence of drugs or alcohol, displays no neurological deficits, has no pain in the middle of the neck and no other painful injuries that could distract from neck pain. If these are all absent, no spinal motion restriction is necessary.

If an unstable spinal column injury is moved, damage may occur to the spinal cord. Between 3 and 25% of SCIs occur not at the time of the initial trauma but later during treatment or transport. While some of this is due to the nature of the injury itself, particularly in the case of multiple or massive trauma, some of it reflects the failure to adequately restrict motion of the spine. SCI can impair the body's ability to keep warm, so warming blankets may be needed.

Initial care in the hospital, as in the prehospital setting, aims to ensure adequate airway, breathing, cardiovascular function, and spinal motion restriction. Imaging the spine to determine the presence of a SCI may need to wait if emergency surgery is needed to stabilize other life-threatening injuries. Acute SCI merits treatment in an intensive care unit, especially injuries to the cervical spinal cord. People with SCI need repeated neurological assessments and treatment by neurosurgeons. People should be removed from the spine board as rapidly as possible to prevent complications from its use.

If the systolic blood pressure falls below 90 mmHg within days of the injury, blood supply to the spinal cord may be reduced, resulting in further damage. Thus it is important to maintain the blood pressure which may be done using intravenous fluids and vasopressors. Vasopressors used include phenylephrine, dopamine, or norepinephrine. Mean arterial blood pressure is measured and kept at 85 to 90 mmHg for seven days after injury.

Various stidies have shown that spinal cord perfusion pressure (SCPP) goals are more closely associated with better neurologic recovery than MAP goals. Some institutions have adopted these SCPP goals and lumbar CSF drain placement as a standard of care. The treatment for shock from blood loss is different from that for neurogenic shock, and could harm people with the latter type, so it is necessary to determine why someone is in shock. However, it is also possible for both causes to exist at the same time. Another important aspect of care is prevention of insufficient oxygen in the bloodstream, which could deprive the spinal cord of oxygen. People with cervical or high thoracic injuries may experience a dangerously slowed heart rate; treatment to speed it may include atropine.

The corticosteroid medication methylprednisolone has been studied for use in SCI with the hope of limiting swelling and secondary injury. As there does not appear to be long term benefits and the medication is associated with risks such as gastrointestinal bleeding and infection its use is not recommended as of 2018. Its use in traumatic brain injury is also not recommended.

Surgery may be necessary, e.g., to relieve excess pressure on the cord, to stabilize the spine, or to put vertebrae back in their proper place. In cases involving instability or compression, failing to operate can lead to worsening of the condition. Surgery is also necessary when something is pressing on the cord, such as bone fragments, blood, material from ligaments or intervertebral discs, or a lodged object from a penetrating injury. Although the ideal timing of surgery is still debated, studies have found that earlier surgical intervention (within 12 hours of injury) is associated with better outcomes. This type of surgery is often referred to as “Ultra-Early”. Sometimes a patient has too many other injuries to be a surgical candidate this early. Surgery is controversial because it has potential complications (such as infection), so in cases where it is not clearly needed (e.g., the cord is being compressed), doctors must decide whether to perform surgery based on aspects of the patient's condition and their own beliefs about its risks and benefits. Recent large-scale studies have shown that patients who do undergo earlier surgery (within 12-24 hours) experience significantly lower rates of life-threatening complications and spend less time in hospital and critical care. However, in cases where a more conservative approach is chosen, bed rest, cervical collars, motion restriction devices, and optionally traction are used. Surgeons may opt to put traction on the spine to remove pressure from the spinal cord by putting dislocated vertebrae back into alignment, but herniation of intervertebral disks may prevent this technique from relieving pressure. Gardner-Wells tongs are one tool used to exert spinal traction to reduce a fracture or dislocation and to reduce motion to the affected areas.

SCI patients often require extended treatment in specialized spinal unit or an intensive care unit. The rehabilitation process typically begins in the acute care setting. Usually, the inpatient phase lasts 8-12 weeks and then the outpatient rehabilitation phase lasts 3-12 months after that, followed by yearly medical and functional evaluation. Physical therapists, occupational therapists, recreational therapists, nurses, social workers, psychologists, and other health care professionals work as a team under the coordination of a physiatrist to decide on goals with the patient and develop a plan of discharge that is appropriate for the person's condition.

In the acute phase physical therapists focus on the patient's respiratory status, prevention of indirect complications (such as pressure ulcers), maintaining range of motion, and keeping available musculature active.

For people whose injuries are high enough to interfere with breathing, there is great emphasis on airway clearance during this stage of recovery. Weakness of respiratory muscles impairs the ability to cough effectively, allowing secretions to accumulate within the lungs. As SCI patients suffer from reduced total lung capacity and tidal volume, physical therapists teach them accessory breathing techniques (e.g., apical breathing, glossopharyngeal breathing) that typically are not taught to healthy individuals. Physical therapy treatment for airway clearance may include manual percussions and vibrations, postural drainage, respiratory muscle training, and assisted cough techniques. Patients are taught to increase their intra-abdominal pressure by leaning forward to induce cough and clear mild secretions. The quad cough technique is done lying on the back with the therapist applying pressure on the abdomen in the rhythm of the cough to maximize expiratory flow and mobilize secretions. Manual abdominal compression is another technique used to increase expiratory flow which later improves coughing. Other techniques used to manage respiratory dysfunction include respiratory muscle pacing, use of a constricting abdominal binder, ventilator-assisted speech, and mechanical ventilation.

The amount of functional recovery and independence achieved in terms of activities of daily living, recreational activities, and employment is affected by the level and severity of injury. The Functional Independence Measure (FIM) is an assessment tool that aims to evaluate the function of patients throughout the rehabilitation process following a spinal cord injury or other serious illness or injury. It can track a patient's progress and degree of independence during rehabilitation. People with SCI may need to use specialized devices and to make modifications to their environment in order to handle activities of daily living and to function independently. Weak joints can be stabilized with devices such as ankle-foot orthoses (AFOs) or knee-ankle-foot orthoses (KAFOs), but walking may still require a lot of effort. Increasing activity will increase chances of recovery.

For treatment of paralysis levels in the lower thoracic spine or lower, starting therapy with an orthosis is promising from the intermediate phase (2-26 weeks after the incident). In patients with complete paraplegia (ASIA A), this applies to lesion heights between T12 and S5. In patients with incomplete paraplegia (ASIA B-D), orthoses are even suitable for lesion heights above T12. In both cases, however, a detailed muscle function test must be carried out to precisely plan the construction with an orthosis.

Spinal cord injuries generally result in at least some incurable impairment even with the best possible treatment. The best predictor of prognosis is the level and completeness of injury, as measured by the ASIA impairment scale. The neurological score at the initial evaluation done 72 hours after injury is the best predictor of how much function will return. Most people with ASIA scores of A (complete injuries) do not have functional motor recovery, but improvement can occur. Most patients with incomplete injuries recover at least some function. Chances of recovering the ability to walk improve with each AIS grade found at the initial examination, e.g., an ASIA D score confers a better chance of walking than a score of C. The symptoms of incomplete injuries can vary and it is difficult to make an accurate prediction of the outcome. A person with a mild, incomplete injury at the T5 vertebra will have a much better chance of using his or her legs than a person with a severe, complete injury at exactly the same place. Of the incomplete SCI syndromes, Brown-Séquard and central cord syndromes have the best prognosis for recovery and anterior cord syndrome has the worst.

People with nontraumatic causes of SCI have been found to be less likely to suffer complete injuries and some complications such as pressure sores and deep vein thrombosis, and to have shorter hospital stays. Their scores on functional tests were better than those of people with traumatic SCI upon hospital admission, but when they were tested upon discharge, those with traumatic SCI had improved such that both groups' results were the same. In addition to the completeness and level of the injury, age and concurrent health problems affect the extent to which a person with SCI will be able to live independently and to walk. However, in general people with injuries to L3 or below will likely be able to walk functionally, T10 and below to walk around the house with bracing, and C7 and below to live independently. New therapies are beginning to provide hope for better outcomes in patients with SCI, but most are in the experimental/translational stage.

One important predictor of motor recovery in an area is presence of sensation there, particularly pain perception. Most motor recovery occurs in the first-year post-injury, but modest improvements can continue for years; sensory recovery is more limited. Recovery is typically quickest during the first six months. Spinal shock, in which reflexes are suppressed, occurs immediately after the injury and resolves largely within three months but continues resolving gradually for another 15.

Sexual dysfunction after spinal injury is common. Problems that can occur include erectile dysfunction, loss of ability to ejaculate, insufficient lubrication of the vagina, and reduced sensation and impaired ability to orgasm. Despite this, many people learn ways to adapt their sexual practices so they can lead satisfying sex lives.

Although life expectancy has improved with better care options, it is still not as good as the uninjured population. The higher the level of injury, and the more complete the injury, the greater the reduction in life expectancy. Mortality is very elevated within a year of injury.

### II. Nerve Graft Fusions

### A. Peripheral Nerve Grafts

The nerve grafts of the present disclosure may be peripheral nerve allografts, autografts or xenografts. Nerve allotransplantation is the transplantation of a nerve to a recipient from a donor of the same species. For example, nerve tissue is transplanted from one person to another. Allotransplantation is a commonly used type of transplantation of which nerve repair is one specific aspect. In contrast, a nerve autograft is a nerve tissue removed from an individual for use in another site in the same individual. Autografts are transplants involving tissue from the same subject as both donor and recipient. Xenografts are transplants between donor and recipients of distinct species.

A nerve allograft is used for the reconstruction of nerve discontinuities in order to support the axonal regeneration across a nerve gap caused by any injury. Nerve tissue may be processed to remove cellular and non-cellular factors such as cells, fat, blood, axonal debris and chondroitin sulfate proteoglycans while preserving the three-dimensional scaffold and basal lamina tubular structure of the nerve. Such nerve allograft only consists of extracellular matrix (ECM), which is sterile and decellularized.

There are three types of peripheral nerves: sensory nerves, which carry sensory information from peripheral organs (e.g., skin) to the central nervous system (responsible for sensation and proprioception); motor nerves-carry information from the central nervous system to peripheral organs (e.g., muscles) (nerve signal activity modulates muscle contraction, thereby enabling movement); mixed nerves containing both sensory and motor fibers.

In a trauma or surgical resection, a nerve can be damaged, which is called a nerve defect. This defect needs to be repaired in order to regain full or partial sensory and motor function. For example, peripheral nerve injury is a major clinical problem and can result in neuropathic pain, which is pain arising as a direct consequence of a lesion or disease affecting the somatosensory system. Damaged nerve fibers continuously excite electric pulses, inducing pain or abnormal sensation dysesthesia. It has been shown that in allograft surgeries, post-operative neuropathic pain was present in some patients, but only if they suffered from this condition pre-operatively. Patients without neuropathic pain before their surgery did not complain about neuropathic pain afterwards. Hence, allograft treatment does not seem to be a risk factor for this specific problem.

### B. Nerve Transplantation

There are several kinds of transplantation techniques. As discussed above, nerve auto-transplantation is transplantation within the same person. However, when there is a large nerve defect, there may be an insufficient number of nerves available for transplantation. The nervus suralis, a nerve from the lower leg, is often used. Consequently, the patient will miss the specific nerve used as an autograft. Therefore, a person's own nerves cannot be used for an unlimited number of times. As such, allografts—nerve material from a genetically distinct subject of the same species—have grown in use over the past several decades. This both avoids additional; injury to the subject and permits multiple surgical interventions on the same subject. Xenografts may also be used.

The transplant surgery generally involves the following steps. First, the surgeon has to prepare the broken nerve. This means the surgeon has to examine the local tissue and resecting scar tissue if needed. The proximal and distal segments of the injured nerves should be debrided to healthy tissue by visual and tactile signs. After that, the surgeon measures the distance between both nerve ends as well as the diameter of the damaged nerve. The surgeon then chooses between a nerve allograft or an autograft, but the procedure is essentially the same. In its most simple sense, this means sutures that connect the graft with the damaged nerve are placed in the epineurium. The present disclosure envisions two approached-bridging transplants and spanning transplants. All-important anatomical structures of the nerve are kept intact.

### C. Selecting and Preparing Nerve Graft Material

There are several factors that help a surgeon decide whether a nerve autograft or an allograft should be chosen above. While the use of nerve autografts has some advantages, such as the lack of immune response by the subject, there are some drawbacks. One is that the surgeon always creates a defect in the donor from where the nerve is taken. Another disadvantage is that when the defect is large, the amount of available autograft material may be insufficient.

Nerve allografts bring a possible solution for some of these problems. Therefore, allografts can be used more often in the same patient than autografts. Studies suggest that nerve allografts work just as well as nerve autografts and are therefore a good alternative to the classic nerve autograft. One adverse effect of nerve allotransplantation is the immunogenic response. Tissue from another human being is used to restore the defect, which can induce an immunogenic response. An immune response against an allograft or xenograft is called transplant rejection. To prevent this rejection, one typically employs immunosuppressive techniques the graft, before it is transplanted into the receiver. Allografts can be processed in such a way that the immune response against the transplant is reduced provoked.

An important feature of graft preparation is that certain structures remain undamaged. The axon is the part of a neuron which conducts electrical impulses. Axons are surrounded by myelin, which contain Schwann cells. Schwann cells improve electrical conduction. Myelin is surrounded by endoneurium, which is a protective sheath of connective tissue. This is surrounded by perineurium and epineurium, of which the latter is the outmost layer of dense connective tissue. When it comes to nerve repair, it is crucial that those layers make a good connection. However, the present disclosure is based on the notion that removal of the extracellular matrix within the epineural space can improve the quality and utility of the remaining structures, i.e., the fascicles and axons disposed therein.

### D. Fusogen Treatment

The present disclosure employs a cell membrane fusogen. These can be of a chemical nature or a biological nature. Examples include the cell-cell fusogenic glyoproteins (e.g., H-alpha 7) and agents such PEG, chitosan, dextran sulfate, N-nonyl. Bromide, calcium and sodium nitrate.

### 1. Cell-Cell Fusogens

Cell-cell fusogens are glycoproteins that facilitate the fusion of cell-to-cell membranes. Cell-cell fusion is critical for the merging of gamete genomes and the development of organs in multicellular organisms. Cell-cell fusion occurs when both actin cytoskeleton and fusogenic proteins properly rearrange across the cell membrane. This process is led by actin-propelled membrane protrusions.

EFF-AFF are the identifiers for type 1 glycoproteins that make up cell-cell fusogens. They were first identified when EFF-1 mutants were found to “block cell fusion in all epidermal and vulval epithelia” in the roundworm, Caenorhabditis elegans. EFF-AFF is a family of type I membrane glycoproteins that act as cell-cell fusogens, named “Anchor cell fusion failure”. Because it was known that EFF-1 mutants successfully fused the anchor cell and (uterine seam) use syncytium to produce a continuous uterine-vulval tube, where these connections failed, AFF-1 mutants were discovered. AFF-1 was deemed necessary for this process in addition to the fusion of heterologous cells in C. elegans. The transmembrane forms of these proteins, like most viral fusogens, possess an N-terminal signal sequence followed by a long extracellular portion, a predicted transmembrane domain, and a short intracellular tail. “A striking conservation in the position and number of all 16 cysteines in the extracellular portion” of EFF-AFF proteins from different nematode species suggests that these proteins are folded in a similar 3D structure that is essential for their fusogenic activity. C. elegans AFF-1 and EFF-1 proteins are essential for developmental cell-to-cell fusion and can merge insect cells. “Thus, FFs comprise an ancient family of cellular fusogens that can promote fusion when expressed on a viral particle.” In the medical field, experiments are done to test for the uses of cell-cell fusogens in axonal nerve repairs and to determine their usefulness with other nerve cells. The current method for nerve repair is suturing the cut ends of nerves. This has a long recovery process, with a low functionality rate for the repaired nerves. When considering cell-cell fusogens as a potential answer, researchers divided these fusogens into two groups based on fusion mechanisms: cell aggregation and membrane modification. One fusogen PEG was found to fit in both groups. It was this fusogen that made restoring nerve cells in humans possible. Once operations were within a certain time frame (12 hours for human nerve repair and 24 hours for sciatic rat treatments), patient recovery was almost successful. With this research, there is potential for repairing human nerve grafts. Some potential uses of cell-cell fusogens studied are cancer vaccines and the regeneration of damaged cells. Additionally, any peripheral nerve in the body could be repaired, and transferred tissues could work as soon as the senses return. Finally, any surgery done on nerves could be repaired as well, thus resulting in a quicker recovery.

### 2. Polyethylene Glycol

The inventors have previously reported (Ghergherehchi et al., 2016; Mikesh et al., 2018a,b) a well-defined protocol for the administration of four pharmaceutical agents in solution, one of which contains the plasmalemmal fusogen PEG (Gefter et al., 1977; Lentz and Lee, 1999; Lentz, 2007; Pontecorvo, 1975) as an adjunct to standard neurorrhaphy for primary repair of PNIs. This protocol results in the immediate reconnection (fusion) of the open axonal ends (PEG-fusion) of many axons in closely apposed proximal and distal ends of singly transected nerves (Bittner et al., 2012; Lore et al., 1999; Mikesh et al., 2018a). The same is true for PEG-fused auto- and allografted nerve segments to repair segmental ablation injuries (Bittner et al., 2015; Mikesh et al., 2018b; Riley et al., 2015). In contrast to tissue repair strategies using neurorrhaphy alone, PEG-fusion as an adjunct to neurorrhaphy also repairs many severed cellular (axonal) processes within the PNS nerve tissue (e.g., rodent sciatic nerve).

In conjunction with neurorrhaphy, PEG-fusion of axons in singly cut PNIs or PEG-fusion of axons in autografts/allografts to repair ablation PNIs in a rat sciatic nerve model produces dramatically improved recovery of coordinated, volitional function as measured by the Sciatic Functional Index (SFI: Bittner et al., 2012; de Medinaceli et al., 1982; Wood et al., 2011). The inventors (Mikesh et al., 2018a,b) have also demonstrated that PEG-fusion results in: (a) restoration of axolemmal and axoplasmic continuity and action potential through-conduction across the coaptation site(s) within minutes, (b) prevention of Wallerian degeneration for many axonal segments distal to the coaptation site(s), (c) preservation of distal NMJs indefinitely and prevention of distal target muscle atrophy, (d) recovery of voluntary function (behavior) that occurs rapidly (days to weeks), often to levels seen in unoperated animals, and to substantially improved quality compared to animals repaired with neurorrhaphy alone, and (e) non-rejection of living allogenic allografts in outbred rats in the non-protected environment of a sciatic peripheral nerve with no immunosuppressive treatments. Furthermore, human clinical cases show that the improved speed and quality of sensory recovery of PEG-fused severed digital nerves are very similar to those reported for recovery of function after PEG-fusion of severed sciatic nerves in the rat sciatic injury model (Bamba et al., 2016b).

The table below from Ghergherehchi et al. (2019) shows an exemplary protocol:

PEG-fusion protocol

| Step # | Technique | Purpose(s) |
|---|---|---|
| Preperation | Trim nerve ends | Prepare nerve ends for [symbol] and PEG |
| | | repair [symbol] fusion |
| 1 - Priming | Irrigation of thew surgical field with [symbol] | Increase [symbol] volume |
| | Ca² [symbol] for 1-2 min | Open cut [symbol] ends |
| | | Expel intracellular membrane- [symbol] |
| 2 - Protection | Administration of 1% methylene [symbol] (M [symbol] | Prevent formation of intracellular [symbol] |
| | antioxidant) in distilled water for 1-2 min [symbol] | [symbol] with PEG-fusion of [symbol] ends |
| | the [symbol] ends | |
| 3 - Coa [symbol] cut | Perform [symbol] | Provide mechanical strength to [symbol] to |
| peripheral nerve ends | | prevent PEG-fused [symbol] |
| | | [symbol] |
| 4 - PEG- [symbol] | Apply 50% w/w 3.35 kDa PEG in distilled | Remove [symbol] cell water to induce [symbol] |
| | water for 1-2 min to the [symbol] | open, [symbol] membranes to [symbol] |
| 5 - Complete | Irrigation of the [symbol] site with [symbol] | Induce vesicle formation to [symbol] any [symbol] |
| membrane repair | volume of [symbol] Ca² [symbol] -containing saline | holes after PEG-induced annealing of the open [symbol] |
| | | [symbol] ends |

[symbol]

indicates data missing or illegible when filed

### III. Kits

Also provided are kits comprising reagents suitable for use in repairing spinal cord injuries. The kits will comprise an allograft and polyethylene glycol. The kits may also comprise additional items such as surgical instruments (scalpel, sutures/needles), containers for mixing PEG solutions and/or for treating allografts, etc. A kit also typically includes a label or packaging insert including a description of the components or instructions for use in vitro, in vivo, or ex vivo, of the components therein.

A kit refers to a physical structure housing one or more components of the kit. Packaging material can maintain the components in a sterile environment and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can also include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, or an electrical storage media such as RAM and ROM or hybrids of these such as magnetic/optical storage media, FLASH memory, hybrids and memory type cards.

### IV. Examples

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

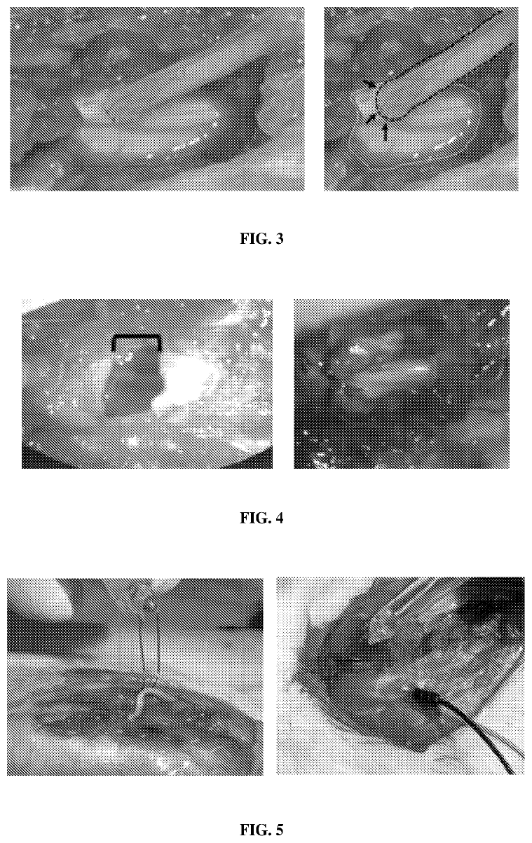

The inventors have successfully developed a surgical protocol in order to carefully expose the spinal cord, induce a contusive or transection type injury, insert and suture peripheral nerve grafts into the spinal cord, and apply PEG-fusion. They have developed two methods of graft insertion: the “bridging” technique and the “spanning” technique (FIG. 2). The “bridging” technique is performed by making a small incision in the spinal cord dura, inserting the peripheral nerve graft into the spinal cord, and securing by sutures (FIG. 3). The “spanning” technique is performed by aspirating damaged spinal tissue to create a visible gap, inserting peripheral nerve grafts into this gap, and securing by sutures (FIG. 4). The bridging technique is less invasive (there is no tissue removal, only a small insertion made into the dura), but is more difficult to get peripheral axons in close contact with spinal axons for fusion. The spanning technique is more invasive but allows for a greater contact between peripheral and spinal axons. For this reason, the inventors are performing both protocols and to test the efficiency of these two techniques.

In addition to testing the efficacy of the two grafting procedures, they are also testing whether PEG-fusion can repair spinal cord injury immediately after damage (acute injury) and 3 weeks after damage (chronic injury). To date, they have successfully measured re-establishment of action potential conduction in vivo in 4/5 animals using the “bridging” repair, and 3/4 in the “spanning repair (FIG. 5). In addition to the in vivo methods, the inventors are currently working to develop an ex vivo testing procedure using a double sucrose gap chamber to get more detailed electrophysiological measures.

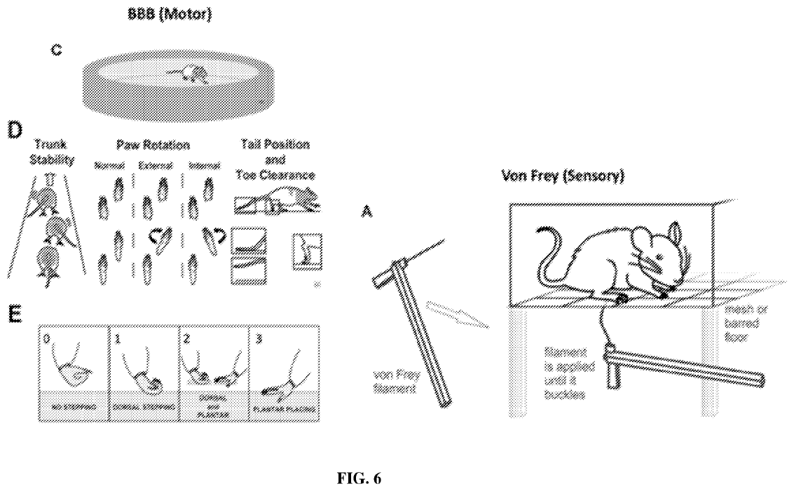

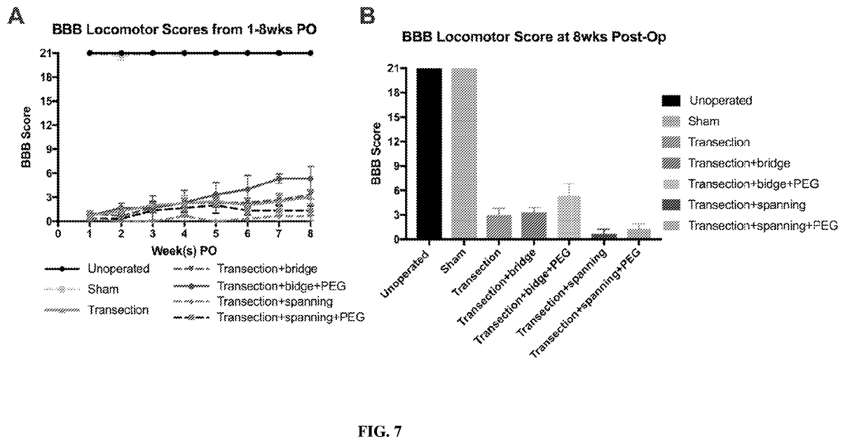

Now that surgical protocols are established, the preliminary data obtained suggests PEG-fusion of peripheral nerve grafts do re-establish connectivity. The inventors have worked to generate animals to analyze functional recovery and will use two tests: the Basso Beattie and Breshnehan (BBB) test for locomotion, and the Von Frey Hair test for sensory recovery (FIG. 6). They will test each animal every week for 12 weeks, determine if PEG groups are superior to controls, which nerve grafting technique is superior, and whether PEG-fusion works for acute and chronic spinal cord injuries. In addition to the weekly testing, animals will need to be cared for four times a day for the duration of the study to ensure proper maintenance of health and bladder expression.

In sum, the inventors have successfully developed a procedure for PEG-fusing peripheral nerve grafts into the spinal cord. Preliminary ephysiological data indicates PEG-fused PNGs restore connectivity in ˜75-80% of animals tested to date using both “bridging” and “spanning” techniques. The process of generating animals and test for recovery of motor and sensory functions after a complete spinal cord transection injury and various treatments is underway. The inventors will compare recovery with and without the use of peripheral nerve grafts and bridging versus spanning grafts to determine the best therapeutic intervention. Due to the nature of the injury (complete transection), any recovery of function is unexpected.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. References

The following references, to the extent that they provide exemplary procedural or other details supplementary to those set forth herein, are specifically incorporated herein by reference.
Bittner et al., J. Neurosci. Res. 90, 967-980 (2012).
de Medinaceli et al., Exp. Neurol. 77 (3), 634-643 (1982).
Bamba et al., J. Trauma Acute Care Surg 81, S177-S183 (2016b).
Mikesh et al., Neurosci. Res. 96, 1223-1242 (2018a).
Mikesh et al., J. Neurosci. Res. 96, 1243-1264 (2018b).
Wood et al., Ann. Anat. 193, 321-333 (2011).
Ghergherehchi et al., J. Neurosci. Methods, 312:1-12 (2019).

1. A method of repairing an injury to a spinal cord nerve in a subject comprising:
(a) providing a peripheral nerve graft or spinal tract nerve graft;
(b) treating said nerve graft with a membrane fusogen such as polyethylene glycol (PEG) solution;
(c) suturing the fusogen-treated nerve graft to the damaged spinal cord such that the fusogen-treated nerve graft bridges or span a region of spinal cord damage or a gap in said spinal cord.

2. The method of claim 1, wherein the fusogen-treated nerve graft bridges around said region of damaged spinal cord.

3. The method of claim 1, wherein the fusogen-treated nerve graft spans a gap in said spinal cord, optionally wherein said method further comprises remove damaged spinal cord tissue to create said gap.

4. The method of claim 1, wherein the peripheral nerve graft is an allograft, an autograft or a xenograft.

5. The method of claim 1, wherein the peripheral nerve graft is derived from a sensory nerve, motor nerve, or a mixed nerve.

6. The method of claim 1, wherein the fusogen is a PEG solution.

7. The method of claim 1, wherein the subject is a human subject.

8. The method of claim 1, wherein the subject is a non-human mammalian subject.

9. The method of claim 1, wherein the spinal cord injury is an acute injury.

10. The method of claim 1, wherein the spinal cord injury is a chronic injury.

11. The method of claim 1, wherein spinal cord injury is to the cervical spinal cord.

12. The method of claim 1, wherein spinal cord injury is to the thoracic spinal cord.

13. The method of claim 1, wherein spinal cord injury is to the lumbar spinal cord.

14. The method of claim 1, wherein spinal cord injury is to the sacral spinal cord.

15. The method of claim 1, wherein injury is to the cervical and thoracic spinal cord, is to the thoracic and lumbar spinal cord, or is to the lumbar and sacral spinal cord.

16. The method of claim 1, wherein the spinal cord injury is a contusion spinal cord injury.

17. The method of claim 1, wherein the spinal cord injury is a transecting spinal cord injury.

18. The method of claim 17, wherein the transecting spinal cord injury is a partial transecting spinal cord injury.

19. The method of claim 17, wherein the transecting spinal cord injury is a complete transecting spinal cord injury.
